(19) United States
(12) Patent Application Publication
(10) Pub. No.: US 2023/0238101 A1
(43) Pub. Date: Jul. 27, 2023

(54) PLATFORM TO DELIVER AN EFFECTIVE TAPERING MODEL

(71) Applicant: OPOS, Inc., San Mateo, CA (US)

(72) Inventors: Justin KROMELOW, Kirkland, WA (US); Rao MIKKILINENI, Cupertino, CA (US); Julie CAREY, San Mateo, CA (US)

(21) Appl. No.: 18/159,637

(22) Filed: Jan. 25, 2023

Related U.S. Application Data
(60) Provisional application No. 63/303,946, filed on Jan. 27, 2022.

(51) Int. Cl. G16H 20/10; G16H 50/20; G16H 80/00

(52) U.S. Cl. CPC G16H 20/10; G16H 50/20; G16H 80/00

(57) ABSTRACT

The present invention relates to a platform for delivering an effective tapering model, comprising: a first set of instructions when executed by the processor triggers the capture and tracking of a patient's data, wherein the data includes clinical interactions at discrete points in time, compare the tracked data with the pre-determined labeled data sets of variables, wherein the data sets include pre-taper patient characteristics, identify a distinction in a time-line, construct a new taper plan by employing one or more supervised machine learning techniques, and recommend at least one potential tapering step for the patient by employing multi-label classification of neural networks.

### FIELD OF INVENTION

The present invention relates to a platform for delivering an effective tapering model specific to a trigger or event in a taper treatment.

### BACKGROUND

Opioids are widely used to manage chronic pain with almost 20 million people being prescribed a 30-day or longer prescription. According to the Center for Disease Control and Prevention (CDC), there are between 9 million and 18 million adults receiving opioids at a greater than recommended dosage. While higher dosages may be justified for some patients under the care of pain specialists, it does represent an increased mortality risk and increased risk of accidental overdose, and hence CDC recommends using the lowest dose of opioids.

Conventionally, an opioid taper is opted to lower the opioid dosage and tapering is conducted in a medically supervised inpatient or outpatient setting. Existing methods are employed by observing the techniques used by pain specialists and combining that information with the patient experience using machine algorithms. However, these methods lack real-time analysis and assessment to deliver customized suggestions at each step of the treatment plan.

Thus, to provide a better, individualized and flexible taper plan based on individual patient requirements, there is a need for an effective and optimized method that can support the patient in a daily program to bring them to a healthier life by providing a successful treatment process and maintain a well-balanced life.

### OBJECTIVES OF THE INVENTION

The primary objective of the present invention is to provide a platform for delivering an effective tapering model to normalize data and determine time continuum variables specific to a trigger or event in a taper treatment.

Another objective of the present invention is to monitor in real-time, within the course of their lifestyle for any triggering taper points and provide personalized treatment plans to reverse chronic conditions.

### SUMMARY

Various embodiments of the present disclosure provide methods and systems for delivering an effective tapering model.

In an embodiment, a computer-implemented method is disclosed. The method comprises receiving an assessment profile of a patient for analyzing the treatment plan for a patient, tracking and collecting the patient's data associated with the patient's profile, comparing the collected data with the pre-determined labeled data sets of variables, identifying one or more distinctions in the timeline; and constructing a taper plan with at least one recommendations for the patient.

In another embodiment, a system is disclosed, the system comprising

a memory for storing software instructions, the software instructions executable by one or more hardware processors of the medical monitoring hub to cause the one or more hardware processors to receive an assessment profile of a patient for analyzing the treatment plan for a patient, track and collect patient's data associated with the patient's profile, compare the collected data with the pre-determined labeled data sets of variables, identify one or more distinctions in the timeline, and construct a taper plan with at least one recommendations for the patient.

### BRIEF DESCRIPTION OF THE DRAWINGS

The disclosed embodiments have other advantages and features which will be more readily apparent from the following detailed description of the invention and the appended claims, when taken in conjunction with the accompanying drawings, in which:

FIG. 1 is an environment of a tapering model delivery platform according to one embodiment;

FIG. 2 is one example embodiment of the present disclosure illustrating a schematic representation of a neural network system, according to one embodiment;

FIG. 3 is a flowchart illustrating an example method, according to one embodiment; and

FIG. 4 is a block diagram illustrating components of an example machine able to read instructions from a machine-readable medium and execute them in a processor (or controller).

### DETAILED DESCRIPTION

The figures and the following description relate to preferred embodiments by way of illustration only. It should be noted from the following discussion, that alternative embodiments of the structures and methods disclosed herein will be readily recognized as viable alternatives and may be employed without departing from the principles of what is claimed.

In order to address the above-noted need for improved methods and systems for detecting and addressing situations involving the improper prescription of medication, improper utilization of prescribed medications, and diversion of prescribed medications to unprescribed uses, the present disclosure is directed to an improved controlled medications usage such as opioid, tracking platform that proposes an effective tapering model. The platform can be utilized to perform various functions, including but not limited to delivering an effective tapering model, collecting and comparing the patient's clinical interactions and/or consumption of resources, at discrete points in time for any trigger in the treatment, and recommending the next potential tapering step for the patient. Each of these functions can be performed separately or in various combinations to address the above-noted and other needs associated with the goal of preventing adverse drug-related events.

With respect to recommending the next step in the taper plan or alternative taper plans, the platform employs one or more supervised machine learning techniques to determine time continuum variables specific to detect an anomaly of a trigger or event in a taper treatment, wherein the variables are contextualized data and are captured by tracking patient's clinical interactions and/or consumption of resources, at discrete points in time. The collected data is then compared with the pre-determined labeled data sets of variables, wherein the data sets include pre-taper patient characteristics. The platform then identifies the distinction in the timelines and constructs a taper plan.

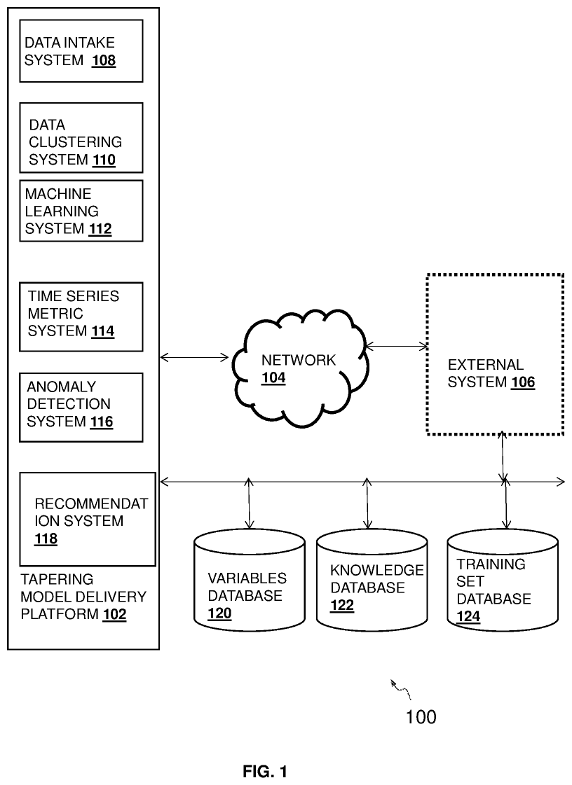

FIG. 1 illustrates an environment 100 of a platform for delivering an effective tapering model to normalize data and determine time continuum variables specific to a trigger or event in a taper treatment. According to an embodiment, the environment comprises a network 104, an external system 106, and a tapering model delivery platform 102 (or platform 102).

The tapering model delivery platform 102 is configured to collect and monitor data related to tracking patient's clinical interactions and/or consumption of resources, at discrete points of time collected in connection with a treatment of a patient and/or data relating to opioid medications that are prescribed to the patient. The tapering model delivery platform 102 may obtain data from multiple external sources, including electronic medical records (EMRs), insurer databases, pharmacy databases, testing lab databases, drug monitoring programs, and/or other suitable data sources.

The tapering model delivery platform 102 may use the obtained data by employing machine learning techniques to normalize data and determine time continuum variables to detect any anomaly in the treatment. e.g. platform 102 can determine whether a patient may be overusing a controlled medication (e.g. an opiate, a benzodiazepine, or amphetamine), determine whether a prescribed treatment plan is over-ordering or under-ordering dosages for their patients; and/or assess the improvement or deterioration in the patient's health.

The tapering model delivery platform 102 compares the collected contextualized data by identifying distinctions in the timelines and constructs a taper plan. The tapering model delivery platform 102 uses a multi-label classification of neural networks to automate the taper process by predicting and recommending the next potential tapering step for the patient.

According to an embodiment, the tapering model delivery platform 102 may build a patient profile along with an opioid usage profile for determining whether a usage profile of a patient is indicative of potential misuse of one or more controlled medications. A machine learning model (ML) or other forms of artificial intelligence (AI) are utilized to generate a potential misuse score or similar measurement of the likelihood that a patient is or has the potential for misusing a controlled substance. In some aspects, laboratory test results from a laboratory that is indicative of a toxicology screen of the patient may be used in conjunction with patient attributes of the patient, to generate the usage profile of the patient. Various features of the usage profile can be utilized with the AI system to determine the likelihood that the patient is or has the potential for misusing a controlled substance. In response to determining that the patient is or has the potential for misusing a controlled substance, notification or report of the patient's potential for misusing a controlled substance can be provided to healthcare professionals in order to assist with the recommended treatment plan of the patient. The tapering model delivery platform 102 may perform additional or alternative tasks without departing from the scope of the disclosure.

According to an embodiment, the tapering model delivery platform 102 may include a data intake system 108, a data clustering system 110, a machine learning system 112, a time series metric system 114, a anomaly detection system 116 and a recommendation system 118.

The data intake system 108 obtains prescription-related information relating to a patient and monitors communications between health care professionals and the patient. The data intake system 108 may output determined instances of interactions and clinical interactions to a data clustering system 110.

The data intake system 108 may generate patient profiles (e.g., patient records) based on the collected data, wherein the profile details can include a patient's name, metadata regarding the patient (e.g. age, sex, weight, height, body fat percentage), prescription-related data obtained by tracking the prescriptions of controlled medications (e.g. opiates, benzodiazepines, amphetamines) and the like.

The data clustering system 110 is configured to receive or collect at least one of the user characteristics in association with the tapering treatment of controlled substances. The data may be normalized for removing bias, before further processing of the computation.

The data clustering system 110 in conjunction with a machine learning system 112 parses the received or collected data and selects at least a data set as a function of at least one variable, wherein the variable is contextualized data identified by tracking patient's clinical interactions and/or consumption of resources at constant intervals.

The machine learning system 112 then generates at least a clustering model containing dataset labels as a function of at least a user pre-taper patient characteristics. The machine learning system 112 may group and/or segment datasets with shared attributes to extrapolate algorithmic relationships. The data clustering system 110 may group data to create clusters that may be categorized by certain classifications and/or commonality labels. The data clustering system 110 may identify commonalities in data and react based on the presence or absence of such commonalities and thereby generate labels to identify clusters of data relating to pre-taper patient characteristics.

Further, the machine learning system 112 includes supervised machine learning techniques such as multivariate logistic regression (MLR), least absolute shrinkage and selection operator-type regression (LASSO), random forest (RF), gradient boosting machine (GBM), and deep neural network (DNN) to determine specific details of the dataset to assign sufficient meaning for variables in the model.

The time series metric system 114 is configured to collect time series data associated with the patient's psychological or physiological attributes of the pre-taper patient characteristics at regular intervals of time, such as after every 5 seconds or 10 seconds.

The anomaly detection system 116 is configured to detect and observe any deviations in the timelines tracked by the time series metric system 114. The anomaly detection system 116 may generate a trigger associated with an abnormal reading, and transmit a notification or alert to the user, the designated person(s) or medical personnel.

The recommendation system 118 is configured to refine the treatment plans, initiatives, and other suggested courses of action based on analysis of data sources. The recommendation system 118 updates and lists the effective and relevant steps of action in the treatment plans in correlation to any anomaly detected.

Further, the tapering model delivery platform 102 may communicate with external system 106 such as user devices, health care applications installed on the user devices, health care system, health care professionals, electronic medical records (EMRs), insurer databases, pharmacy databases, testing lab databases, and/or prescription drug monitoring programs, as well as another computing device (s), systems, data sources, applications, and platforms, via a network 104.

Network 104 may include, for example, one or more of the Internet, Wide Area Networks (WANs), Local Area Networks (LANs), analog or digital wired and wireless telephone networks (e.g., a public switched telephone network (PSTN), Integrated Services Digital Network (ISDN), a cellular network, and Digital Subscriber Line (DSL)), radio, television, cable, satellite, or any other delivery or tunneling mechanism for carrying data. Network 104 may include multiple networks or subnetworks, each of which may include, for example, a wired or wireless data pathway. Network 104 may include a circuit-switched network, a packet-switched data network, or any other network able to carry electronic communications (e.g., data or voice communications.) For example, network 104 may include networks based on the Internet protocol (IP), asynchronous transfer mode (ATM), the PSTN, packet-switched networks based on IP, X.25, or Frame Relay, or other comparable technologies and may support voice using, for example, VoIP, or other comparable protocols used for voice communications. Network 104 may include one or more networks that include wireless data channels and wireless voice channels. Network 104 may be a wireless network, a broadband network, or a combination of networks including a wireless network and a broadband network.

The external system 106 may refer to a collection of one or more computing devices, including client user devices and/or server devices that are used in connection with a healthcare organization (e.g. one or more hospitals, doctor offices, etc.). In embodiments, an external system 106 may include an EMR data store. An EMR data store may include one or more databases that store and/or index electronic medical records. A respective electronic medical record may store or reference patient data of a respective patient of healthcare organization. An electronic medical record may include a patient identifier, one or more physician identifiers that indicate the respective physicians of a patient, physician notes relating to the patient, prescription data indicating treatments that were prescribed to a patient, test results of the patient, and the like.

According to an embodiment, the tapering model delivery platform 102, is in communication with one or more databases to retrieve and store the associated data. For example, a variable database 120 may include information describing a user's daily habits, previous treatment plans, and previous triggers such as a medication that didn't alleviate the user's symptoms or a particular food that still causes the user distress. A knowledge database 122 may include data entries reflecting one or more expert submissions of data such as may have been submitted according to any process, including without limitation by using the graphical user interface. The knowledge database 122 may include one or more fields generated by the data clustering system 110, such as without limitation fields extracted from one or more documents as described above. Training set database 124 may contain training sets pertaining to different categories and classifications of information, including training set components which may contain sub-categories of different training sets. In an embodiment, at least a server may select at least a training set by classifying at least a user input data to generate at least a classified user variable containing at least a label of the dataset.

The aforementioned modules may be used and interconnected in various combinations with one another. The modules may be in communication with a central server. In an embodiment, the modules may reside within a computer-readable medium, a server, a computer, a phone, a mobile device, a wearable device, or other electronic devices.

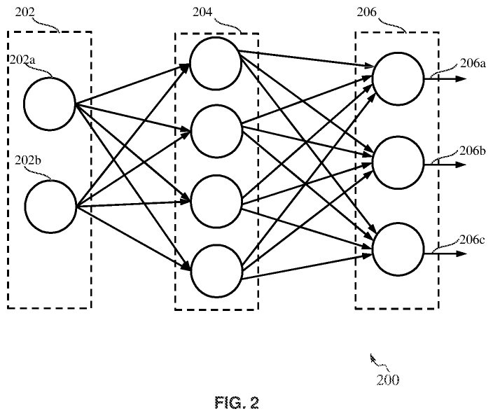

FIG. 2 is one example embodiment of the present disclosure illustrating a schematic representation of a neural network system 200 embedded in the machine learning system 112 of the platform 102 for automating the taper process by predicting and recommending the next potential tapering step for the patient using multi-label classification. System 200 may be embedded with a processor. System 200 may be a framework for a plurality of machine learning algorithms that may operate in tandem and process the data received. System 200 may be configured to receive training in order to process the complex data. The training may be carried out by feeding the system 200 with sample data without being programmed by task-specific rules.

A typical system 200 comprises an input layer 202, a computing layer 204, and an output layer 206. The input layer 202 may include example data sets or standard data sets. In one embodiment, the input layer 202 may include two nodes, a first node 202a representing a pre-taper patient characteristics data input and a second node 202b representing a time series data input. The input layer 202 with the data from the first node 202a and the second node 202b may be transmitted to the computing layer 204. The computing layer 204 in system 200 may be a layer in between the input layer 202 and the output layer 206, where steps and algorithms are included to process the data received and provide an output through an activation function. The computing layer 204 may be set up such that, the computing layer 204 may process data. Once the computing layer 204 processes the data received from the first node 202a and the second node 202b, an output or a result may be generated. The output generated may be routed to the output layer 206. The output layer 206 may include multiple nodes based on the type of information to be communicated. In an embodiment, the output layer 206 may include a first output node 206a, a second output node 206b, and a third output node 206c. In an embodiment, the first output node 206a may provide the output pertaining to the current status of the treatment plan, the second output node 206b may provide the output pertaining to the treatment suggestions and the third output node 206c may provide the output pertaining to health management of the patient. The output from the output layer 206 may be transmitted to a communication device for communicating the results obtained to an external device 106.

In an embodiment, system 200 may be trained by providing examples. This configuration ensures that system 200, in the subsequent iteration, may process similar information faster, thereby improving the processing or computation speed of platform 102. For example, system 200 can access labeled examples and their respective variables stored on databases and can then use the pre-determined labeled data sets of variables, wherein the data sets include pre-taper patient characteristics to learn (i.e., train) what variables deviate from the collected data. After the initial training, system 200 can then be used to probabilistically predict labels of datasets, given their variables, estimates the probability or statistics that the deviated value, and provide the desired result to the user as per the requirement.

In an embodiment, system 200 may automatically compute and provide results to the patient or health care professional based on the treatment plan. In an embodiment, the system 200 may automatically compute and provide results including but not limited to a current health status, a possibility of a trigger, suggestions of treatment and health management, and the like to the patient or health care professional.

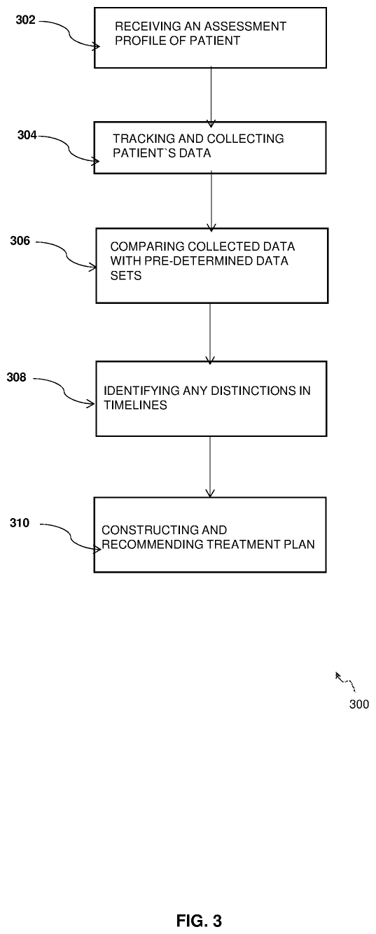

FIG. 3 is a flow diagram 300 representing the method to deliver an effective tapering model by identifying any deviating tapering points in the timeline of the tapering treatment according to an embodiment. Platform 102 is configured to perform a method, using a processor, that includes receiving 302 an assessment profile for analyzing the treatment plan for a patient, at 304 tracking and collecting patient's data such as clinical interactions and/or consumption of resources and the like, at 306 comparing the collected data with the pre-determined labeled data sets of variables, wherein the data sets include pre-taper patient characteristics, at 308 identifying any distinctions in the timelines and at 310 constructing a taper plan and recommending the next step.

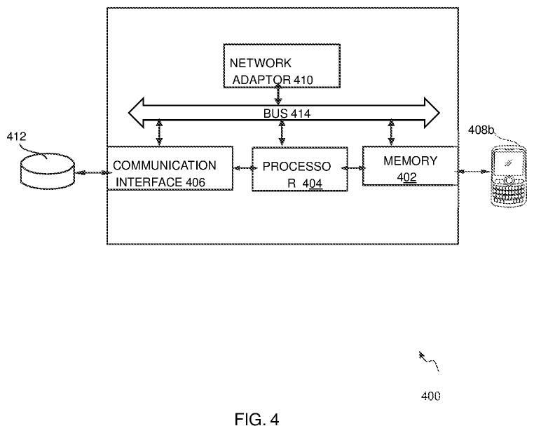

FIG. 4 is an example embodiment of the present disclosure illustrating a system 400 for delivering an effective taper model. In an example embodiment, the system 400 includes a database 412, a communication interface 406, a network interface 410, a processor 404, and a memory 402 for processing information.

Database 412 may be a record of any form, relevant to processing the information by platform 102. In an embodiment, the database 412 may include the variable database 120, the knowledge database 122, and the training set database 124 of FIG. 1.

The communication interface 406 may enable system 400 to communicate with one or more users through the communication device 408b of FIG. 1. Optionally, system 400 may also receive input from the communication interface 406 is directly coupled to the server 400 or via the communication device 408b. The system 400 is also shown to be communicably coupled to an output display (not shown in figures), including but not limited to a cathode ray tube (CRT), an LCD screen, and a laptop screen for displaying information to the user. The communication interface 406 is capable of communicating with the communication device 408b, examples of which may include but are not limited to, wired, wireless cell phone networks, Wi-Fi networks, terrestrial microwave networks, or any form of Internet. In an embodiment, the communication device 408b may also act as a display device for displaying the results data or the output computed by system 400.

The server 400 can communicate with one or more networks such as a local area network (LAN), a general wide area network (WAN), and/or a public network (e.g., the Internet) via network adapter 410. As depicted, network adapter 410 communicates with the other components of the computer system/server 400 via bus 414. It should be understood that although not shown, other hardware and/or software components could be used in conjunction with the computer system/server 400. Examples include, but are not limited to: microcode, device drivers, redundant processing units, external disk drive arrays, RAID systems, tape drives, and data archival storage systems, etc.

Memory 402 is a storage device embodied as one or more volatile memory devices, one or more non-volatile memory devices, and/or a combination of one or more volatile memory devices and non-volatile memory devices, for storing micro-contents information and instructions. The memory 402 may be embodied as magnetic storage devices (such as hard disk drives, floppy disks, magnetic tapes, etc.), optical magnetic storage devices (e.g., magneto-optical disks), CD-ROM (compact disc read-only memory), CD-R (compact disc recordable), CD-R/W (compact disc rewritable), DVD (Digital Versatile Disc), BD (Blu-ray® Disc), and semiconductor memories (such as mask ROM, PROM (programmable ROM), EPROM (erasable PROM), flash ROM, RAM (random access memory), etc.). In an embodiment, memory 402 may also be cloud-based storage.

Processor 404 is communicably coupled with memory 402 and the communication interface 406. Processor 404 is capable of executing the stored machine executable instructions in memory 402 or within processor 404 or any storage location accessible to processor 404. The processor 404 may be embodied in a number of different ways. In an example embodiment, processor 404 may be embodied as one or more of various processing devices, such as a coprocessor, a microprocessor, a controller, a digital signal processor (DSP), processing circuitry with or without an accompanying DSP, or various other processing devices including integrated circuits such as, for example, an application specific integrated circuit (ASIC), a field programmable gate array (FPGA), a microcontroller unit (MCU), a hardware accelerator, a special-purpose computer chip, or the like. The processor 404 performs various functionalities of the system 400.

Various embodiments described above may be implemented in software, hardware, application logic or a combination of software, hardware, and application logic. The software, application logic, and/or hardware may reside on at least one memory, at least one processor, an apparatus, or, a non-transitory computer program product. In an example embodiment, the application logic, software, or instruction set is maintained on any one of various conventional computer-readable media. A computer-readable medium may comprise a computer-readable storage medium that may be any media or means that can contain or store the instructions for use by or in connection with an instruction execution system, apparatus, or device, such as a computer.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, thereby enabling others skilled in the art to best utilize the present invention and various embodiments with various modifications as are suited to the particular use contemplated.

What is claimed is:

1. A system for delivering an effective tapering model in a taper treatment, the system comprising: a memory for storing software instructions, the software instructions executable by one or more hardware processors of the medical monitoring hub to cause the one or more hardware processors to:
receive an assessment profile of a patient for analyzing the treatment plan for a patient;
track and collect patient data associated with the patient's profile;
compare the collected data with the pre-determined labeled data sets of variables;
identify one or more distinctions in the timeline; and
construct a taper plan with at least one recommendation for the patient.

2. The system as recited in claim 1, wherein the data sets include pre-taper patient characteristics.

3. The system as recited in claim 1, wherein the patient's data is collected and monitored by tracking the patient's clinical interactions or consumption of resources, at discrete points of time during the treatment.

4. The system as recited in claim 1, wherein the system, further employs machine learning techniques to normalize data and determine time continuum variables to detect any anomaly in the treatment.

5. The system as recited in claim 1, wherein the system, further uses a multi-label classification of neural networks to automate the taper process by predicting and recommending the next potential tapering step for the patient.

6. The system as recited in claim 1, wherein the system, further builds a patient profile and an opioid usage profile for determining whether a usage profile of a patient is indicative of potential misuse of one or more controlled medications.

7. The system as recited in claim 1, wherein the system, further generates a potential misuse score or similar measurement of the likelihood for misusing a controlled substance by the patient.

8. The system as recited in claim 1, wherein the system, further generates a trigger associated with an abnormal reading, and transmits a notification or alert to the user, the designated person(s), or medical personnel.

9. The system as recited in claim 1, wherein the system, further updates and lists the effective and relevant steps of action in the treatment plan in correlation to the detected anomaly.

10. The system as recited in claim 1, wherein the system, further communicates with external systems such as user devices, health care applications installed on the user devices, health care system, health care professionals, electronic medical records (EMRs), insurer databases, pharmacy databases, testing lab databases, and/or prescription drug monitoring programs, as well as other computing devices, systems, data sources, applications, and platforms, via a network.

11. A computer-implemented for delivering an effective tapering model in a taper treatment, the method comprises:
receiving an assessment profile of a patient for analyzing the treatment plan for a patient;
tracking and collecting patient data associated with the patient's profile;
comparing the collected data with the pre-determined labeled data sets of variables;
identifying one or more distinctions in the timeline; and
constructing a taper plan with at least one recommendation for the patient.

12. The method as recited in claim 11, wherein the data sets include pre-taper patient characteristics.

13. The method as recited in claim 11, wherein the patient's data is collected and monitored by tracking the patient's clinical interactions or consumption of resources, at discrete points of time during the treatment.

14. The method as recited in claim 11, wherein the method, further employs machine learning techniques to normalize data and determine time continuum variables to detect any anomaly in the treatment.

15. The method as recited in claim 11, wherein the method, further uses a multi-label classification of neural networks to automate the taper process by predicting and recommending the next potential tapering step for the patient.

16. The method as recited in claim 11, wherein the method, further builds a patient profile and an opioid usage profile for determining whether a usage profile of a patient is indicative of potential misuse of one or more controlled medications.

17. The method as recited in claim 11, wherein the method, further generates a potential misuse score or similar measurement of the likelihood of misusing a controlled substance by the patient.

18. The method as recited in claim 11, wherein the method, further generates a trigger associated with an abnormal reading, and transmits a notification or alert to the user, the designated person(s) or medical personnel.

19. The method as recited in claim 11, wherein the method, further updates and lists the effective and relevant steps of action in the treatment plan in correlation to the detected anomaly.

20. The method as recited in claim 11, wherein the method, further communicates with an external system such as user devices, health care applications installed on the user devices, health care system, health care professionals, electronic medical records (EMRs), insurer databases, pharmacy databases, testing lab databases, and/or prescription drug monitoring programs, as well as other computing devices, systems, data sources, applications, and platforms, via a network.
